(19) United States
(12) Patent Application Publication
(10) Pub. No.: US 2023/0281163 A1
(43) Pub. Date: Sep. 7, 2023

(54) SYSTEMS AND METHODS FOR DATA ANALYSIS AND VISUALISATION

(71) Applicant: Tercen Data Analytics Limited, Waterford (IE)

(72) Inventors: Faris NAJI, Waterford (IE); Martin ENGLISH, Waterford (IE); Alexandre MAUREL, Waterford (IE)

(21) Appl. No.: 18/178,306

(22) Filed: Mar. 3, 2023

Related U.S. Application Data
(60) Provisional application No. 63/316,683, filed on Mar. 4, 2022.

(51) Int. Cl. G06F 16/16; G06F 3/0486; G06F 40/177

(52) U.S. Cl. CPC G06F 16/168; G06F 3/0486; G06F 40/177

(57) ABSTRACT

Systems and methods are described herein for a a machine, such as a cytometer, configured to make a measure a series of measurements and to export characterising data, including in real-time, for each of those measurements; and a computer configured to receive that characterising data. With the characterising data, the computer is configured to perform the steps of executing datasteps in a datastep workflow using the characterising data and, optionally, to recognise predetermined patterns, including those indicative of a medical condition, during execution of the datasteps.

### CROSS-REFERENCE TO RELATED APPLICATION(S)

This is application is a nonprovisional application claiming benefit to U.S. Patent Application No. 63/316,683, filed on Mar. 4, 2022, which is incorporated herein by reference in its entirety.

### FIELD OF THE DISCLOSURE

Various embodiments of the present disclosure pertain generally to systems and methods for data analysis and visualization.

### BACKGROUND OF THE INVENTION

Cytometry is the measurement of the characteristics of cells. Variables that can be measured by cytometric methods include cell size, cell count, cell morphology (shape and structure), cell cycle phase, DNA content, and the existence or absence of specific proteins on the cell surface or in the cytoplasm. Cytometry is used to characterize and count blood cells in common blood tests such as the complete blood count. In a similar fashion, cytometry is also used in cell biology research and in medical diagnostics to characterize cells in a wide range of applications associated with diseases such as cancer and AIDS.

Image cytometry is the oldest form of cytometry. Image cytometers operate by statically imaging a large number of cells using optical microscopy. Prior to analysis, cells are commonly stained to enhance contrast or to detect specific molecules by labeling these with fluorochromes. Traditionally, cells are viewed within a hemocytometer to aid manual counting. Since the introduction of the digital camera, in the mid-1990s, the automation level of image cytometers has steadily increased. This has led to the commercial availability of automated image cytometers, ranging from simple cell counters to sophisticated high-content screening systems.

Due to the early difficulties of automating microscopy, the flow cytometer has since the mid-1950s been the dominating cytometric device. Flow cytometers operate by aligning single cells using flow techniques. The cells are characterized optically or by the use of an electrical impedance method called the Coulter principle. To detect specific molecules when optically characterized, cells are in most cases stained with the same type of fluorochromes that are used by image cytometers. Flow cytometers generally provide less data than image cytometers, but have a significantly higher throughput.

The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY OF THE INVENTION

According to certain aspects of the present disclosure, the systems and methods described herein a system is provided comprising a machine, such as a cytometer, configured to make a measure a series of measurements and to export characterising data, including in real-time, for each of those measurements; and a computer configured to receive that characterising data. With the characterising data, the computer is configured to perform the steps of executing datasteps in a datastep workflow using the characterising data and, optionally, to recognise predetermined patterns, including those indicative of a medical condition, during execution of the datasteps.

The datasteps in the datastep workflow are configured by displaying a workflow diagram containing an element indicative of a data set different from the characterising data and elements indicative of subsequent datasteps of a workflow that a user has configured; and displaying in the workflow diagram under the control of a user the connection of a functional element in the workflow whereby at least one workflow datastep is located downstream of the connected functional element, the functional element being associated with a series of instructions. When executed, the method perform the steps of characterising those downstream datasteps by identifying configuration settings of those datasteps including settings related to which data headers are used in those datasteps; and mapping those identified data headers to data headers of the characterising data so that, when executed, the datasteps are equivalent but use the characterising data;

In a further such system, the computer is configured to display a projection of the characterising data in the body of a table, the projection having been configured by displaying a data workflow diagram containing an element indicative of the real-time characterising data; creating a new step in the data workflow (hereafter a new ‘datastep’) using the real-time characterising data; displaying in the datastep window a table having a primary row header, a primary column header, at least one nestled row header, at least one nestled column header and a table body; and displaying the selection by dragging and dropping by a user of data headers of the characterising data on to the primary and/or nestled row and column headers, whereby the projection of the characterising data in the body of the table is accordingly to the selected headers.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 shows, schematically, a system for medical diagnosis, according to techniques presented herein;

FIG. 2 shows, schematically, a data workflow diagram of a graphical user interface, according to techniques presented herein;

FIGS. 3 to 7 show, schematically, datastep windows for configuring datasteps of the data workflow of FIG. 1, according to techniques presented herein;

FIG. 8A to 8E shows, schematically, a modified data workflow diagram of a graphical user interface, according to techniques presented herein.

### DETAILED DESCRIPTION OF THE INVENTION

Cell sorters are flow cytometers capable of sorting cells according to their characteristics. The sorting is achieved by using technology similar to what is used in inkjet printers. The fluid stream is broken up into droplets by a mechanical vibration. The droplets are then electrically charged according to the characteristics of the cell contained within the droplet. Depending on their charge, the droplets are finally deflected by an electric field into different containers.

Cytometers are therefore capable of producing in real-time substantial quantities of data which, if analysed quickly and accurately, this can lead to the identification of adverse medical conditions.

Data analysis is the process of cleaning, manipulating, inspecting, and modelling raw data with the view to gain insight or discover meaning in the raw data. In the modern world, data analysis is becoming a driving force in decision making for businesses and governments worldwide. With this being the case, it is necessary that any analysis performed can be inspected and tweaked, as any minor errors in any step of an analysis process can perpetuate throughout the analysis leading to potentially incorrect results and as a consequence, incorrect conclusions being drawn from the raw data.

Traditionally in data analysis, raw data is uploaded into analysis software, the raw data may then be manipulated through the use of various functions before plotting either the raw data or the manipulated data to provide visualization, for example, a graph.

In conventional data analysis software, data is typically presented in a table or matrix and functions can be performed on some or all of the rows/columns of the data to gain insight, producing yet further tables or matrices containing manipulated data. With large data sets, and/or in situations where the analysis of the data requires multiple complex steps, it can become difficult to keep track of what has been done. There exists a need for improved visualization and control over the steps in data analysis processes.

Furthermore, it is often the case that data analysis and manipulation is completed before the result of the said analysis is plotted as a graph or other visual. This method is limiting, however, as it tends to limit the data analysis to a step-by-step path from raw data to result. This traditional way of working, therefore, misses the possibility of finding unexpected links between data sets and/or variables within a data set. Further, it lends itself away from making speculative analyses due to the end-goal orientated nature of the process. This could lead to insights being missed. Therefore, there exists a need for faster more intuitive systems and methods for data analysis that moves away from the goal orientated way of thinking, whilst remaining structured and understandable. Understandable here relating to how easy it is to tell from looking at the data analysis software what steps have been carried out to which data set.

An additional problem in the field of data analysis is that of scalability. With large data sets and multiple steps of data analysis to be executed, large amounts of computing power is required to perform the analysis. Especially in the case that each new step in the analysis depends on the results of one or more previous steps. There is a need in the art of a more computationally and storage efficient data analysis package to deal with such a situation.

Some examples of data analysis include an analysis method for large and/or complex biological data sets from molecular biology experiments comprising importing data in a table data structure, comparing data points, calculating an optimized data representation and displaying the representation.

Some examples of data analysis include techniques facilitating using flow graphs to represent a data analysis program in a cloud-based system for open science collaboration and discovery. In an example, a system can represent a data analysis execution as a flow graph where vertices of the flow graph represent function calls made during the data analysis program and edges between the vertices represent objects passed between the functions. In another example, the flow graph can then be annotated using an annotation database to label the recognized function calls and objects. In another example, the system can then semantically label the annotated flow graph by aligning the annotated graph with a knowledge base of data analysis concepts to provide context for the operations being performed by the data analysis program.

Systems and methods described herein aims to provide a system for medical diagnosis comprising a machine, such as but not limited to a cytometer, which is configured to receive a series of medical samples and to export in real-time characterising data for each of those samples; and a computer configured to receive and analyse that characterising data.

In the following description, like features are given like numerals.

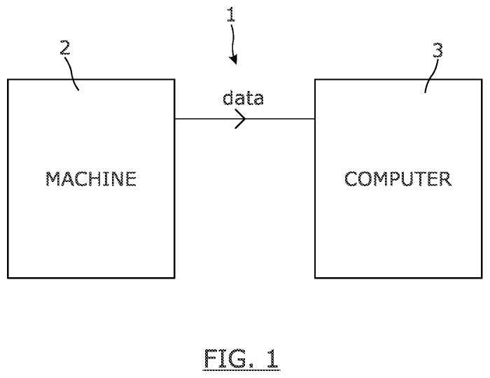

FIG. 1 shows, schematically, a system 1 for medical diagnosis comprising a machine, such as a cytometer, configured to receive a series of medical samples and to export in real-time characterising data for each of those samples; and a computer 2 configured to receive and analyse that characterising data which is configured as described below (that characterising data illustrated as containing data with headers A to D).

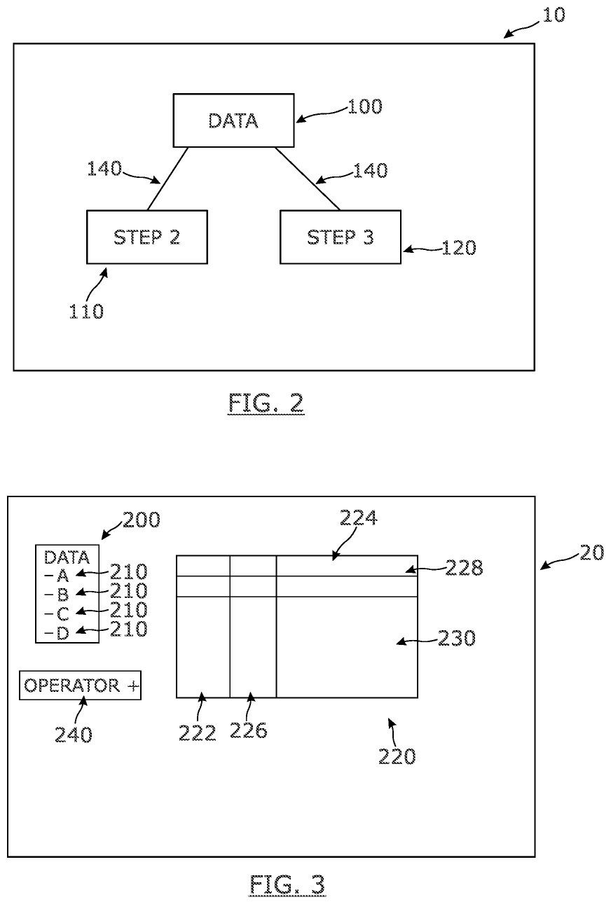

FIG. 2 shows a data workflow diagram 10 of a graphical user interface of computer 2 having a rectangular box 100 labelled ‘DATA’ which is the representative of real-time characterising data uploaded from machine 2 and which, in the context of a data workflow, can be considered to be an initial or first datastep 100). Also shown in the data workflow diagram are second and third rectangular boxes 110, 120 labelled ‘STEP 2’ & ‘STEP 3’ which representative of further datasteps in a data workflow. The workflow diagram contains connecting lines, which indicate the respective relationships of datastep ‘STEP 2’ and datastep ‘STEP 3’ to the uploaded data set/first data step ‘DATA’. As will be further described below, the datastep ‘STEP 2’ is used to visualize the data of the uploaded data set/first data step 100 and the third datastep (step 3) is an operand resulting from an operation applied to the uploaded data set/datastep ‘DATA’.

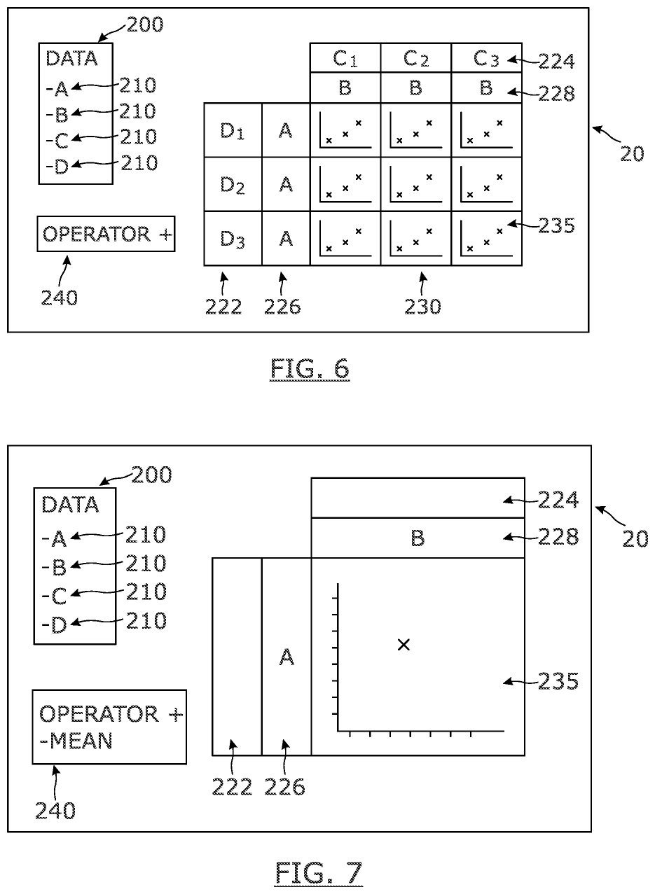

FIG. 3 shows an unconfigured datastep window 20 of a graphical user interface which can be used by a user to configure a datastep based on datastep ‘DATA’/the uploaded data set. The datastep window contains a rectangular box 200 labelled ‘DATA’ which contains a list of selectable headers of data A to D 210 which is that have been extracted from datastep ‘DATA’. The datastep window further contains a blank table 220 having a primary row header 224, a primary column header 226, a nestled row header 228, a nestled column header 226 and a table body 230. These might alternatively be referred to as a row zone 220, a column zone 224, a Y axis zone 226, and X axis zone 228 and a plot area 230. To visualize data of datastep ‘DATA’, a user can select headers of data 210 (labelled A to D) by dragging and dropping selected headers on to the primary and/or nestled row and column headers. Such selection results in the projection of data accordingly to the selected headers in the body of the table as exemplified below in FIGS. 3 to 5. Also shown in the datastep window is a rectangular box 240 labelled ‘Operator+’ which when selected reveals a list of available data operations which may be applied to the data available to the datastep, and thus is an operation selection menu 240, again as exemplified below in FIG. 6.

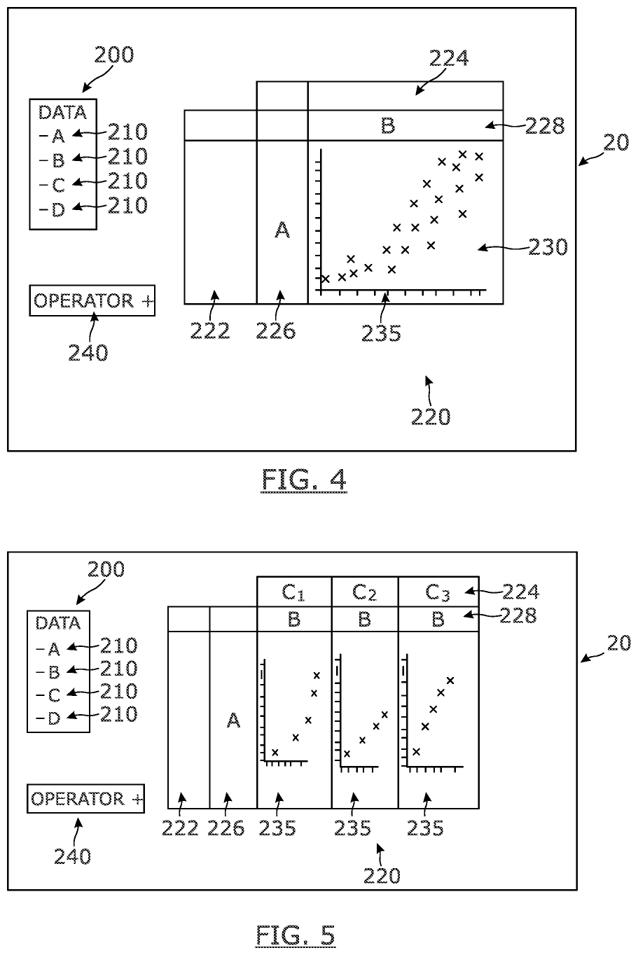

FIG. 4 shows a datastep window after a first exemplary user configuration for datastep ‘STEP 2’ provides, as mentioned, a visualising of data of datastep ‘DATA’ located upstream of datastep ‘STEP 2’ in the data workflow diagram. Specifically, datastep window 20 is show after the user has dragged header ‘A’ into nestled column header/y axis zone 226 and dragged header ‘B’ into nestled row header/X axis zone 228. In this example, A is a dependent variable and B is an independent variable. As a result of the user selecting headers by positioning headers A & B 210 in the table 220 in this way, header A data is plotted against header B data in the body of the table 230.

FIG. 5 shows a datastep window after a second exemplary user configuration for datastep ‘STEP 2’ providing an alternative visualising of data of datastep ‘DATA’. As with the example of FIG. 3, datastep window 20 is shown after the user has dragged header ‘A’ into nestled column header/y axis zone 226 and dragged header ‘B’ into nestled row header/X axis zone 228. In addition, the datastep window 20 is further show after the user has dragged header ‘C’ into the primary row header/row zone 224. In this example, A is a dependent variable, B is an independent variable, C is a sample name and D is a run number. I.e. a measurement is taken of A against B, for sample C and repeated D times. As a result of this user configuration, data of header A is plotted against data of header B for each sample C. This results in a plurality of plots 235 in the plot area 230, one for each of the samples measured.

FIG. 6 shows a datastep window after a third exemplary user configuration for datastep ‘STEP 2’ providing a further alternative visualising of data of datastep ‘DATA’. As with the example of FIG. 4, datastep window 20 is shown after the user has dragged header ‘A’ into nestled column header/y axis zone 226, dragged header ‘B’ into nestled row header/X axis zone 228 and dragged header ‘C’ into the primary row header/row zone 224. In addition, the datastep window 20 is further show after the user has dragged header ‘D’ into the primary column header/column zone 222. In addition, the datastep window 20 is further show after the user has dragged header ‘D’ into the primary row header/row zone 224 so as to cause the projection to present data from each run separately. I.e. one for each instance of D for each of the samples measured.

FIG. 7 shows a datastep window after user configuration for datastep ‘STEP 3’, providing, as mentioned, an operand resulting from an operation applied to the uploaded data set/ datastep ‘DATA’. As with the example of FIG. 3, datastep window 20 is shown after the user has dragged header ‘A’ into nestled column header/y axis zone 226 and dragged header ‘B’ into nestled row header/X axis zone 228. In addition, the datastep window 20 is further shown after the user has selected the operator ‘MEAN’ using the operation menu 240 as a result of which the mean of header A data is plotted against the mean of header B data in the body of the table 230. Note, as the data projection in the body of the table is updated after each user selection, the display of the body may first be that of FIG. 3 before selection of the operator ‘MEAN’ and changing to that of FIG. 6.

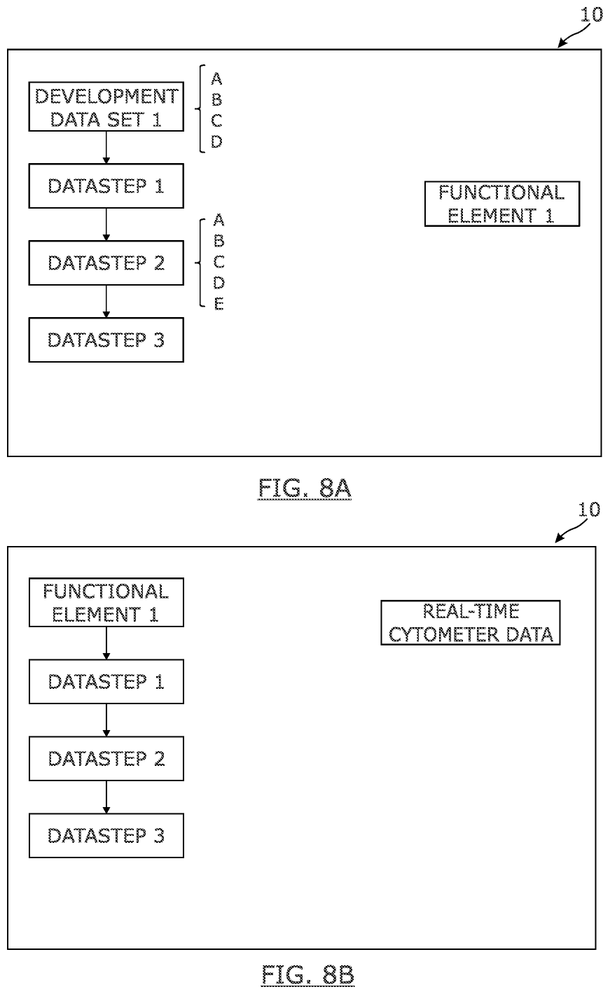

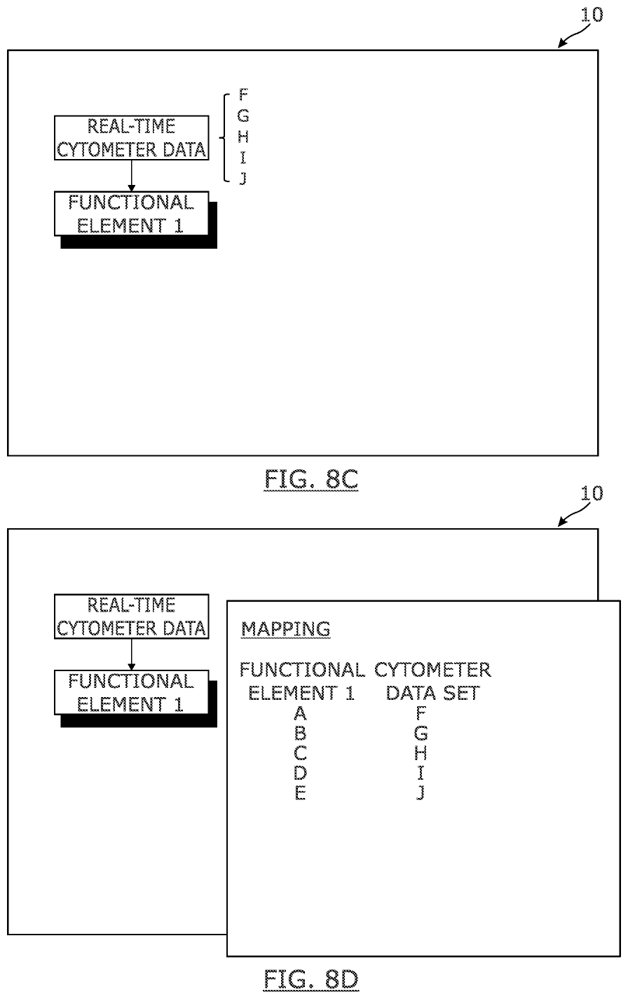

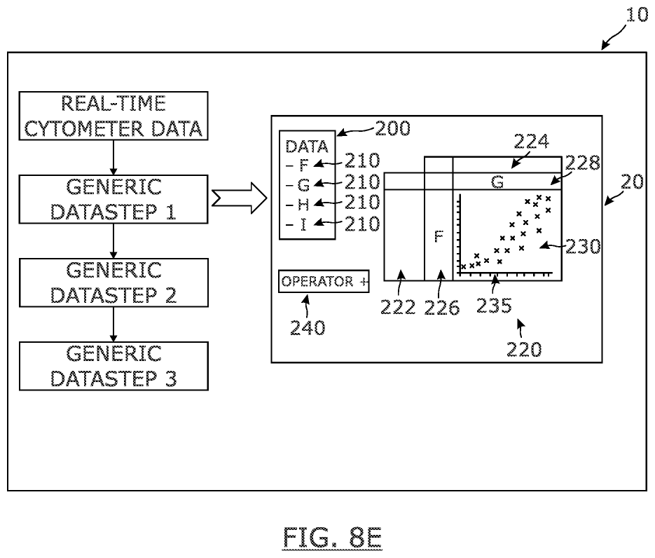

FIGS. 8A to 8E show, schematically, a data workflow diagram of a graphical user interface, according to techniques presented herein. Similar in type to that illustrated in FIG. 1 and with datasteps of the type illustrated in FIGS. 1 to 6, FIG. 8A shows a data workflow diagram with a workflow including a Development Data Set 1 (with data headers A to D) and datasteps 1 to 3. Datastep 1 is configured for a data projection involving data headers A to D. Datastep 2 involves the execution of an operator which creates derivative data E which is not part of the 1^{st} reference dataset. Lastly, Datastep 3 is configured for a data projection involving data headers A to E. The graphical user interface also displays inactive element Functional Element 1.

FIG. 8B shows the data workflow diagram after a user has disconnected Development Data Set 1 from the workflow and instead replaced it with Functional Element 1, connected upstream of Datasteps 1 to 3. Furthermore, the functional element is associated with a series of instructions which, when executed, perform the step of characterising downstream data steps including identifying data headers and operations used in those downstream datasteps and configuration setting of those downstream datasteps (e.g., those which determine the format of the datastep projections). This characterising information is stored in code associated with Functional Element 1. This code, which may be considered to be a datastep signature, may be exported in a file for subsequent reimportation.

FIG. 8C shows a data workflow diagram after a user has executed the method of Functional Element 1 (indicated by the shadowing), disconnected Development Data Set 1 from the workflow and replaced it with Real-time Cytometer Data 2 (with has data identified by data headers F to J). Connection of Real-time Cytometer Data 2 initiates data header mapping whereby headers A to E utilised by datasteps 1 to 3 as identified by ‘Functional Element 1’ are mapped to data headers F to J of the Real-time Cytometer Data.

As shown in FIG. 8D, the graphical user interface presents the user with a mapping window with a preliminary mapping of the headers A to E utilised in Datasteps 1 to 3 and identified by Functional Element 1 mapped to data headers F to J of Real-time Cytometer Data 2. A preliminary mapping may be conducted automatically based on common data header type, typically data values, data header similarly, etc., manually by the user or, preferably, a combination of both. The graphical user interface further provides for selection of Functional Element 1 by the user to reveal a generic representation of Datasteps 1 to 3, as shown in FIG. 8E, and, for example, when connected to Real-time Cytometer Data, selection of Generic Datastep 1 would reveal a datastep window configured in the same manner as that of FIG. 1 but with data from Uploaded Data Step 2.

In summary, Functional Element 1 provides a generic representation or abstraction of data workflow steps—which can be thought of as a workflow transformation—such that they may be designed, saved (exported and reimported), and used with alternate datasets, all in the guise of a data workflow object which is readily manipulated by a user.

In the context of the system 1 of FIG. 1, the machine 2 is configured to receive a series of medical samples and to export in real-time characterising data for each of those samples and the computer 2 configured as described above. I.e. the projections of real-time characterising data (i.e. cytometer data) are configured using the data header selection of FIGS. 3 to 7. Also, the configuration of the data steps used to analyse the real-time characterising data exported by the machine 2 is done offline with a reference data set before being applied to the real-time characterising data as illustrated in FIGS. 8A to 8E. Predetermined patterns indicative of a medical condition during execution of the datasteps can be automatically recognised and an operator warned accordingly.

In another system, such as a CNC machine, where the machine is capable of affecting the nature of that being measured, the machine may be configured to initiate remedial steps if a predetermined pattern is recognised. I.e. the machine may remedy and oversized dimension of a member or a surface anomaly, the CNC machine may be configured to remove offending material.

What is claimed is:

1. A computer system comprising:
a machine configured to make a measure a series of measurements and to export characterising data for each of those measurements; and
a computer configured to receive that characterising data and to perform the steps of:
executing datasteps in a datastep workflow using the characterising data, the datasteps in the datastep workflow having been configured by:
displaying a workflow diagram containing an element indicative of a data set different from the characterising data and elements indicative of subsequent datasteps of a workflow that a user has configured; and
displaying in the workflow diagram under a control of a user a connection of a functional element in the workflow whereby at least one workflow datastep is located downstream of the connected functional element, the functional element being associated with a series of instructions which, when executed, perform the steps of:
characterising those downstream datasteps by identifying configuration settings of those datasteps including settings related to which data headers are used in those datasteps; and
mapping those identified data headers to data headers of the characterising data so that, when executed, the datasteps are equivalent but use the characterising data.

2. The computer system of claim 1, wherein the configuration of the datasteps in the datastep workflow further includes the step of exporting a configuration file including the datastep characterisation.

3. The computer system of claim 1, wherein the configuration of the datasteps in the datastep workflow further includes the step of exporting a configuration file including the datastep characterisation.

4. The computer system of claim 1, wherein during datastep configuration, the step of identifying data headers used in the downstream datasteps includes identifying headers of derivative data created from the data set different from the characterising data in those downstream datasteps.

5. The computer system of claim 4, wherein datasteps equivalence includes creating corresponding derivative data from the characterising data.

6. The computer system of claim 1, wherein identifying configuration settings includes identifying data operations done in those stream datasteps.

7. The computer system of claim 6, wherein datastep equivalence includes applying the identified operations to the characterising data.

8. The computer system of claim 1, wherein the datastep characterisation includes relational algebra which is reapplied to the characterising data.

9. A computer system comprising:
a machine configured to make a measure a series of measurements and to export characterising data for each of those measurements; and
a computer configured to receive that characterising data and perform the steps of:
displaying in real-time a projection of the characterising data in a body of a table, the projection having been configured by:
displaying a data workflow diagram containing an element indicative of the real-time characterising data;
creating a new step in the data workflow (hereafter a new ‘datastep’) using the real-time characterising data;
displaying in a datastep window a table having a primary row header, a primary column header, at least one nestled row header, at least one nestled column header and a table body; and
displaying a selection by dragging and dropping by a user of data headers of the characterising data on to the primary and/or nestled row and column headers, whereby the projection of the characterising data in the body of the table is accordingly to the selected headers.

10. The computer system of claim 9, wherein the computer is configured to apply an operation to at least some of the characterising data to produce derivative data which is part of the projection.

11. The computer system of claim 9, wherein the computer is configured to apply permutations of multiple operations to at least some of the characterising data to produce derivative data which is part of the projection.

12. The computer system of claim 9, wherein the new data is created using relational algebra.

13. The computer system of claim 9, wherein the machine is configured to make a measure a series of measurements and to export characterising data for each of those measurements in real-time.

14. The computer system of claim 9, wherein the machine is configured to recognise predetermined patterns in the characterising data.

15. The computer system of claim 14, wherein the computer is configured to warn a user if a predetermined pattern is recognised.

16. The computer system of claim 14, wherein the machine is configured to make a measure a series of measurements of medical samples, and wherein recognising predetermined patterns in the characterising data is of a medical condition.

17. The computer system of claim 14, wherein the machine is a cytometer.

18. The computer system of claim 14, wherein the machine is configured to initiate remedial steps if a predetermined pattern is recognised.
